(19) United States
(12) Patent Application Publication
(10) Pub. No.: US 2015/0201881 A1
(43) Pub. Date: Jul. 23, 2015

(54) METHOD AND DEVICE FOR ASSISTING IN THE ASSESSMENT OF RHEUMATISM TREATMENTS

(71) Applicant: Siemens Aktiengesellschaft, Munich (DE)

(72) Inventors: Stefan Förtsch, Erlangen (DE); Karsten Hiltawsky, Schwerte (DE); Rainer Kuth, Hoechstadt (DE); Lars Lauer, Neunkirchen (DE); Dominik Paul, Bubenreuth (DE); Sebastian Schmidt, Weisendorf (DE); Susanne Schmolke, Erlangen (DE)

(21) Appl. No.: 14/413,453

(22) PCT Filed: Jun. 13, 2013
(86) PCT No.: PCT/EP2013/062210

§ 371 Date: Jan. 8, 2015

(30) Foreign Application Priority Data
Jul. 10, 2012 (DE) 10 2012 211 995.7

(51) Int. Cl. A61B 5/00; A61B 5/0275; A61B 5/026

(52) U.S. Cl. CPC A61B 5/4848; A61B 5/0263; A61B 5/4509; A61B 5/0275; A61B 5/4528; A61B 2576/02

(57) ABSTRACT

A method is disclosed for determining analysis information supporting a user assessing the effect of a biological agent on the treatment of a rheumatic disease. The method includes, subsequent to starting the administration of the biological agent: recording a first morphological magnetic resonance imaging record of at least one target joint using a first magnetic resonance sequence; recording a second magnetic resonance imaging record showing the blood flow through the target joint by perfusion imaging using a second magnetic resonance sequence; determining at least a first measuring parameter related to the morphology of the joint by analysis of the first magnetic resonance imaging record and at least a second measuring parameter describing the blood flow in the target joint by analysis of the second magnetic resonance imaging record; and automatic determination of the analysis information from the first and the second measuring parameter.

### PRIORITY STATEMENT

This application is the national phase under 35 U.S.C. §371 of PCT International Application No. PCT/EP2013/062210 which has an International filing date of Jun. 13, 2013, which designated the United States of America, and which claims priority to German patent application DE 102012211995.7 filed Jul. 10, 2012, the entire contents of each of which are hereby incorporated herein by reference.

### FIELD

At least one embodiment of invention generally relates to a method for determining analysis information assisting a user assessing the effectiveness, in particular the continued administration, of a biological agent for treating a rheumatic disease.

### BACKGROUND

Rheumatoid arthritis RA is a chronic inflammatory disease of the joints with a clinical progression that is difficult or even impossible to predict. Although rheumatoid arthritis frequently starts in the small joints of the hands and feet, it can lead to serious skeletal changes and the destruction of the affected joints. Rheumatoid arthritis therefore has a significant effect on all aspects of life and is therefore very restricting for those with the disease.

Various symptoms are generally considered when identifying rheumatoid arthritis so the diagnosis is generally based on case history, clinical symptoms, abnormal laboratory test results and x-ray images. The DAS (disease activity score), in particular the DAS 28, has been developed to assess the severity of the disease. In this 28 joints are considered, with the DAS 28 taking account of parameters determined mainly by the physician or patient during the course of an examination, specifically the number of pressure-sensitive joints, the number of swollen joints, the erythrocyte sedimentation rate and the patient's own estimation of the state of the disease. Such a value is therefore not only characterized by the subjective impressions of physician and patient but also takes into account symptoms that can only be observed clinically.

In recent years examination options based on magnetic resonance imaging have also repeatedly been proposed in the prior art. For example other scores or analysis values have been proposed, which describe different morphologically visible effects, for example the so-called RAMRIS scores, as mentioned in the article “OMERACT Rheumatoid Arthritis Magnetic Resonance Imaging Studies. Core Set of MRI Acquisitions, Joint Pathology Definitions, and the OMERACT RA-MRI Scoring System” by Mikkel Østergaard et al., J. Rheumatol 2003; 30:1358-6. This proposes various sequences for morphological magnetic resonance imaging for the diagnosis and assessment of rheumatoid arthritis, on the basis of which certain Ramris scores can be determined for symptoms clearly defined there, in particular changes to the synovium (synovitis), bone edema and bone erosion, as also described in the article by Mikael Boesen et al., “MRI quantification of rheumatoid arthritis: Current knowledge and future perspectives”, European Journal of Radiology 71 (2009) 189-196. However the diagnostic validity of the Ramris scores is disputed and only relates to certain partial effects so the Ramris scores are seldom used in clinical practice.

Biological agents particularly have proven effective for the treatment of rheumatoid arthritis. These include in particular the group of TNF-α inhibitors (anti-tumor necrosis factor alpha). These biological agents allow the suppression of disease activity in patients who do not respond to conventional treatment methods, for example DMARD therapy. However biological agents are extremely expensive.

It should be assumed here that some patients receive the biological agent even though they could also be treated satisfactorily without said medication. It is however difficult to discontinue the medication again as it is unclear whether or not there would be a deterioration in the disease without the biological agent. It is frequently observed that there is a remission in the disease after administration of the biological agent so that clinical symptoms disappear almost completely for example. Nevertheless subclinical symptoms, which can therefore only be detected using dedicated measuring methods, can continue and may not be picked up by the DAS for example.

Until now no procedure has been known in the prior art which allows a comprehensive, reliable assessment of the effect of biological agents on rheumatoid arthritis and in particular indicates whether there is a risk of recurrence.

### SUMMARY

At least one embodiment of the invention is directed to a method for determining analysis information, which allows an assessment relating to the effect and/or discontinuation of a biological agent, in particular of TNF-α inhibitors, for rheumatoid arthritis.

According to an embodiment of the invention, the method includes at least the following, after starting administration of the biological agent in a method for determining analysis information, the method comprising:

recording a first, morphological magnetic resonance image data record of at least one target joint using a first magnetic resonance sequence;

recording a second magnetic resonance image data record showing the blood flow through the target joint by perfusion imaging using a second magnetic resonance sequence;

determining at least one first measuring parameter relating to the morphology of the joint by analysis of the first magnetic resonance image data record and at least one second measuring parameter describing the blood flow through the target joint by analysis of the second magnetic resonance image data record; and

automatically determining the analysis information from the first and second measuring parameters.

In addition to the method, at least one embodiment of the present invention also relates to an analysis device, which is configured to perform at least one embodiment of the inventive method. Such an analysis device can comprise a storage facility, a computation facility, in particular a processor, and corresponding hardware and software components, which implement steps of at least one embodiment of the inventive method electronically in an automatic manner. As mentioned above, it is particularly preferable for at least one embodiment of the inventive method to operate completely automatically, in particular in relation to the analysis and the determination of the analysis information, as this produces standardized and readily comparable data.

If steps, for example also assessments and/or evaluations, which are included in the further calculations, are to be performed manually and/or if other manual inputs are desired, the analysis device can include an input facility, for example a keyboard and/or a mouse. A display facility, for example a monitor, is generally expedient so that the results, in particular the analysis information, can also be displayed. Of course it is also conceivable to transmit the analysis information to external devices by way of a suitable communication connection in order to store or further process it there.

The analysis device can be part of a magnetic resonance facility. In other words it is conceivable to embody a magnetic resonance facility in such a manner that it is already possible to perform the necessary steps for determining the analysis information on the magnetic resonance facility. For example the analysis device can be integrated in a control facility of the magnetic resonance facility or can correspond thereto. It is further conceivable to incorporate the analysis device in an image archiving and analysis system, an information system or the like.

### BRIEF DESCRIPTION OF THE DRAWINGS

Further advantages and details of the present invention will emerge from the example embodiments described in the following and with reference to the drawings, in which:

FIG. 1 shows a sequence of an embodiment of the inventive method, and

FIG. 2 shows an inventive analysis device as part of a magnetic resonance facility.

### DETAILED DESCRIPTION OF THE EXAMPLE EMBODIMENTS

At least one embodiment of the invention is therefore not only based on the knowledge that information about the effect of the biological agent and the state of the disease in respect of the administration of the biological agent can be determined by way of a magnetic resonance examination of at least one target joint but also that the skillful combining of perfusion imaging and morphological imaging allows an excellent analysis in respect of the effectiveness of the biological agent.

According to at least one embodiment of the invention therefore not only are morphological effects such as bone edema, bone erosion, changes to the cartilage and/or synovium considered but a second measuring parameter describing the blood flow through the target joint is also determined as a further biomarker. The blood flow through the target joint provides an indication of the state of inflammation of the target joint. Only by combining the at least one first measuring parameter with the at least second measuring parameter in the analysis information is it possible to obtain a reliable and helpful statement which allows a user, in particular a physician, to make an assessment in respect of the effectiveness of the biological agent.

It should be emphasized here that at least one embodiment of the inventive method does not aim to provide a diagnostic statement with which a physician can comply but simply to assist the physician by way of an in particular automatic and standardized analysis of measurement data, namely magnetic resonance image data, which supplies analysis information which can usefully assist a physician assessing the effect of the biological agent and only has a diagnostic significance when interpreted, in particular together with further examination results. The assessment of the analysis information in respect of diagnostic statements is therefore not part of at least one embodiment of the inventive method, which only deals with the recording and analysis of physical and technical measurement data.

Generally therefore the analysis information can particularly advantageously be used by a physician when assessing the effectiveness of a biological agent, as achieved by skillfully combining different magnetic resonance imaging methods which focus specifically on this question. For example once a physician has analyzed the magnetic resonance examination and obtained the analysis information, it is possible to evaluate the probability of the recurrence of a rheumatic disease after discontinuing a biological agent, in particular a TNF alpha inhibitor. Although the perfusion and morphological imaging methods and therefore the at least one first measuring parameter or the at least one second measuring parameter alone do not have sufficient significance in respect of the effectiveness of the biological agent, it has proven that the advantageous combining of results of the measuring methods can provide a relevant statement correlated to the effectiveness of the biological agent.

An analysis value can expediently be determined as the analysis information. Such analysis values, often known in the medical field as scores, are accepted by the relevant users as established ways for the physician to assess states of a patient. Provision can be made for example for an analysis value within a defined scale, for example a scale from zero to ten, to be determined as analysis information. Generally the analysis information, in particular the analysis value, can be output to the user, it being expedient also to provide an interpretation aid for this purpose.

In a further embodiment of the present invention, provision can be made for the first magnetic resonance sequence used to be a T2-weighted sequence with fat saturation and/or a STIR sequence, in particular for analysis in respect of a measuring parameter describing bone edema, and/or a gadolinium-enhanced, three-dimensional T1-weighted spin-echo or gradient-echo sequence, in particular for analysis in respect of a measuring parameter describing changes to the synovium and/or bone erosion. Such morphological magnetic resonance imaging methods are already known in principle in the prior art and are also set out for example in the article by Mikael Boesen et al. cited in the introduction. They can advantageously also be used in the context of at least one embodiment of the present invention, although other morphological imaging sequences that are known in principle in the prior art are also conceivable.

Provision can further be made for the second magnetic resonance sequence used to be a sequence that measures the progression of a contrast agent in the cartilage of the target joint and/or a sequence using arterial spin labeling. A contrast agent bolus is generally injected during perfusion imaging, the contrast agent diffusing into the cartilage and being washed out again there. The time constants of these processes can then be measured for example by continuously recording magnetic resonance images of the same slice over a measuring time of for example 5 minutes, preferably approx. one minute. It is however also conceivable to perform a perfusion measurement without administering a contrast agent, if appropriate methods, preferably arterial spin labeling (ASL), are used.

It should be pointed out here that static perfusion measurements are also known, which can be used in the context of at least one embodiment of the present invention, in contrast to the dynamic perfusion measurements set out above. With these a contrast agent bolus, for example Gd-GPPA or a blood pool agent, is also injected into the patient at the start of the examination. The circulation of the contrast agent through the target joint, for example through the hand, is then measured with the T1 relaxation time being significantly shortened compared with blood for example. If there are no problems with the blood/tissue barrier, all the molecules of the contrast agent remain in the bloodstream. However if there are blood/tissue barrier problems, the contrast agent passes into the tissue and can be readily identified there due to the short T1 relaxation time.

It should also be noted here that any joints affected by the disease can be considered as target joints. For example it is conceivable to perform examinations on the wrist and/or on metacarpophalangeal joints (MCP).

It is expedient for the magnetic resonance sequences to be implemented as part of a single measuring protocol. Provision can therefore be made for all the measurement data recorded for all the magnetic resonance image data records to be recorded in the context of at least one embodiment of the present invention (in which process a number of first and/or a number of second magnetic resonance image data records can of course also be recorded) to be combined in an automated manner in one measuring protocol. Such combining of measuring sequences and intermediate steps, for example instructions for administering a contrast agent and the like, in one measuring protocol, in which process the specific recording parameters can therefore also be predetermined, is particularly useful in respect of the standardization and comparability of the results, in particular of the analysis information. Therefore a certain workflow is predetermined, which in principle should be performed in an identical manner.

It is particularly advantageous here if the in particular automatic analysis of the magnetic resonance image data records and/or the determination of the analysis information take(s) place as part of the measuring protocol. The measuring protocol therefore serves to control the entire workflow up to the determination of the analysis information. It is expedient anyway to determine the first measuring parameter and/or the second measuring parameter automatically, in particular based on a segmentation of anatomical structures of the target joint. It is however also conceivable to interpose manual analysis steps, for example the definition of an ROI or the like, but this is less preferable in respect of a desired comparability between different examinations.

In order to allow automation of the determination of the measuring parameters and therefore to configure the analysis process in a uniform manner for different examinations, an automatic classifier can be used as a program means for example. It is also conceivable to use an image-guided algorithm, which independently identifies the relevant points in the magnetic resonance image data records and further analyzes them or marks them for a physician. An analysis tool therefore results in particular in respect of image regions located or segmented in a standardized analysis, said analysis tool being able to perform the analysis in a standardized manner and therefore being able to determine the measuring parameters in a standardized manner.

As mentioned above, standardization is also assisted by the automation of the recording processes for the magnetic resonance image data records, in particular in the context of a measuring protocol. In order therefore to allow uniform and simple creation of the magnetic resonance image data records, the setting of the different recording parameters, which are decisive for the quality of the magnetic resonance images, can be set by means of suitable automated predeterminations. In the case of perfusion imaging for example this facilitates the evaluation of the time constant, as the time can be established in an automated manner and the relevant time constant can be calculated.

It should be noted here that during an at least partially manual analysis to determine the first and/or second measuring parameter, support algorithms and evaluation aids known from the prior art can also be supplied, for example the overlaying of displayed magnetic resonance image data with further information and/or comparison image data, which can be retrieved for example from a database.

The first measuring parameter determined can be a measuring parameter describing bone erosion and/or bone edema and/or changes to the cartilage and/or changes to the synovium and/or a Ramris score.

Bone erosion is bone damage, the cause of which can be found in rheumatoid arthritis. Typical radiological findings are for example subchondral osteoporosis, destruction of the surrounding bone, ankyloses and joint misalignment (buttonhole deformity, swan's neck deformity, ulnar deviation).

Changes to the cartilage can be measured by measuring the thickness of the cartilage layers in the region of the target joint. A comparison value or comparison measurement can be used here, which indicates the thickness of the cartilage layer in a healthy target joint. It is also possible to assess a change in thickness in the current patient him/herself, in particular if prior measurements are available, which will be examined in greater detail below. It is of course also conceivable to take into account other cartilage changes.

Cartilage nourishment problems can result in a deterioration of the matrix between the chondrocytes. This is referred to as cartilage demasking. The surface becomes rough, restricting the function of the target joint, so that an arthrosis can develop. Cartilage disease can also occur at the kneecap, which can cause problems at a relatively early age, even in children. In the ribcage strange calcifications can occur at the boundary between bony and cartilaginous rib, which are also referred to as chondrocalcinosis.

Recurring polychondritis is a further, fairly rare disease, which is associated with generally spasmodic but sometimes also long-term inflammation of the cartilage. Chondrosis is an expression of degenerative changes in the cartilage. In spinal disks it can result in a narrowing of the distance between one or more vertebral segments compared with the other vertebral segments, without sclerosing of the end plates of the vertebrae. The height of the intervertebral spaces decreases. With block vertebrae the intervertebral space disappears. Chondrosis involving the underlying bone is referred to as osteochondrosis. If small pieces of cartilage become detached from the mass of cartilage, it is referred to as osteochondritis dissecans.

The synovium is a membrane within a joint. In magnetic resonance imaging it is shown in the manner of a type of artifact, as the susceptibility is different at both sides. Provision can be made here for the shape of the synovium to be compared with standard shapes using a database, in particular a shape library. Standard deviations can be documented automatically and stored as the first measuring parameter.

However it is particularly preferable to use a Ramris score as defined in the articles cited in the introduction, its determination also being able to be automated, in particular by using segmentation and comparison methods. This provides a clearly defined basis, with the determination resulting at least in principle from already known studies.

As a development of at least one embodiment of the present invention, provision can further be made for at least one magnetic resonance image data record of the target joint recorded at an earlier time to be taken into account, in particular as part of a comparison, and/or at least one standard value and/or standard image data record for the analysis of the magnetic resonance image data records. Other data can therefore also be retrieved.

It is advantageous for example for a first magnetic resonance image data record and a second magnetic resonance image data record to be recorded before the administration of the biological agent in order to be able to be used as comparison data records in the context of the analysis. Similarly it is possible to have available the magnetic resonance image data records of different examinations of the inventive type, which were recorded during the course of the therapy, so that the history of the patient can also be mapped to a certain degree by way of measuring parameters. This can be expressed in a comparison with the original, untreated state of the disease as well as in the manner of a trend, if magnetic resonance image data or intermediate parameters determined therefrom is/are available for different times over the duration of the treatment.

Also if there is such a storage mechanism for the history, a progression of the at least one first and the at least one second measuring parameter can be determined and assessed, with the same applying for the analysis information. This means that benefit does not necessarily (only) result from the comparison of first and second magnetic resonance image data records recorded at different times for the determination of the analysis information but also separately from the determination of the same, for example as a further criterion in the assessment of the success of the treatment.

In addition to further information taken into account in the context of the analysis in the form of magnetic resonance image data records (or other image data records recorded using other modalities) of the patient, it is also conceivable, as described above, to take into account standard values and/or standard image data records in the context of the analysis.

Also such data originating for example from healthy people and/or from healthy regions of the current patient can be used as important comparison information in the context of the analysis, for example if a change is to be assessed or a measuring parameter or other analysis parameters are to be given as a rate, in other words as a relative value. For example the provision of standard image data records makes it possible to compare the thickness of a cartilage with normal cartilage thickness, shapes of the synovium with standard shapes and the like.

Also models showing permitted changes can be used in the inventive method and provide useful aids for an in particular automatic analysis. As mentioned above, it can be extremely expedient for the assessment of the success of the treatment if a magnetic resonance image data record recorded before starting the administration of the biological agent is used, in particular first and second magnetic resonance image data records recorded respectively using the at least one first magnetic resonance sequence and the at least one second magnetic resonance sequence before starting the treatment, which can serve as comparison data records.

It should however be pointed out that in principle it is also possible to perform at least one embodiment of the inventive method without such a comparison, as it also allows subclinical effects within the joints, which have a clear influence on the probability of recurrence, to be observed and assessed, as described above.

Already existing, known products, in particular analysis software, can also be used in the context of at least one embodiment of the present invention to allow further automation. Software packages for example are already known for perfusion measurements allowing analysis for example by determining time curves automatically and analyzing them in relation to time constants. One example of such a product is known by the name “tissue 4D” and can also be used in the context of the present invention. Software packages, which segment anatomical features, can also be used correspondingly in the context of the present invention, for example if the cartilage, synovium and the like are to be defined in the morphological first magnetic resonance image data record.

In addition to the method, at least one embodiment of the present invention also relates to an analysis device, which is configured to perform at least one embodiment of the inventive method. Such an analysis device can comprise a storage facility, a computation facility, in particular a processor, and corresponding hardware and software components, which implement steps of at least one embodiment of the inventive method electronically in an automatic manner. As mentioned above, it is particularly preferable for at least one embodiment of the inventive method to operate completely automatically, in particular in relation to the analysis and the determination of the analysis information, as this produces standardized and readily comparable data.

If steps, for example also assessments and/or evaluations, which are included in the further calculations, are to be performed manually and/or if other manual inputs are desired, the analysis device can include an input facility, for example a keyboard and/or a mouse. A display facility, for example a monitor, is generally expedient so that the results, in particular the analysis information, can also be displayed. Of course it is also conceivable to transmit the analysis information to external devices by way of a suitable communication connection in order to store or further process it there.

The analysis device can be part of a magnetic resonance facility. In other words it is conceivable to embody a magnetic resonance facility in such a manner that it is already possible to perform the necessary steps for determining the analysis information on the magnetic resonance facility. For example the analysis device can be integrated in a control facility of the magnetic resonance facility or can correspond thereto. It is further conceivable to incorporate the analysis device in an image archiving and analysis system, an information system or the like.

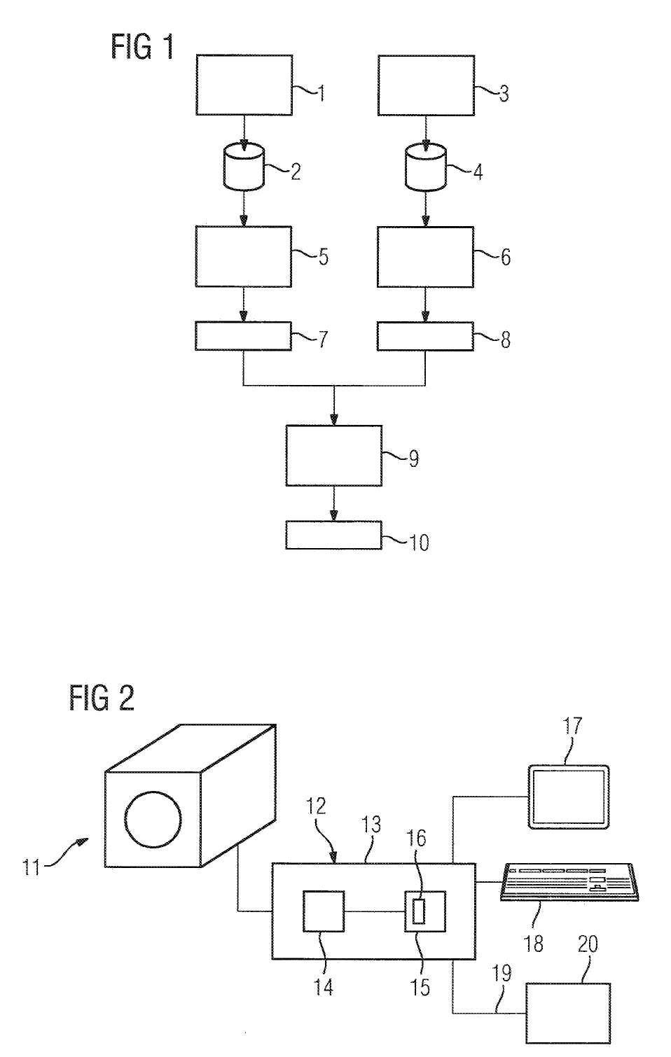

FIG. 1 shows a basic outline of the sequence of an embodiment of the inventive method.

In a step 1 at least one first magnetic resonance image data record 2 is recorded using at least one first magnetic resonance sequence. The at least one first magnetic resonance data record 2 is a morphological magnetic resonance image data record; in other words the structure of at least one target joint can be identified therein, in particular with regard to the bone involved, the cartilage and/or the synovium. Of course, if target joints in different regions of the human body are to be considered, a number of first magnetic resonance image data records can also be recorded for a number of target joints and it is also possible to record a number of magnetic resonance image data records for one or each target joint, each highlighting different anatomical features and so on.

In the present example embodiment, provision is made for recording T1-weighted image data before and after the intravenous administration of gadolinium contrast agent in a first magnetic resonance image data record. Such first magnetic resonance image data records are particularly suitable for allowing the identification of bone erosion and changes to the synovium, in particular synovitis, therein. A T2-weighted magnetic resonance with fat saturation is recorded as a further first magnetic resonance image data record; this can be used for example to deduce bone edema.

The magnetic resonance sequences used to record the first magnetic resonance image data records 2 are part of a single measuring protocol, which predetermines the corresponding recording parameters for the magnetic resonance facility in a fixed manner. Also part of this measuring protocol is the use of at least one second magnetic resonance sequence, which is used to record at least one second magnetic resonance image data record 4 in a step 3. This is a perfusion magnetic resonance image data record 4, from which information can therefore be derived relating to the blood flow through the target joint, in particular through the cartilage. In the present exemplary embodiment dynamic perfusion is used for this purpose.

In the present example embodiment, the measuring protocol also extends to the analysis of the image data records 2, 4, which in this instance takes place in steps 5, 6 and in this example embodiment takes place in a fully automated manner. In other words the image data is analyzed by hardware components and/or software components implemented in an analysis device, it being possible for the analysis device to be implemented for example as the control facility of a magnetic resonance facility.

In step 5 provision can be made specifically for example for different parts of the target joint first to be segmented by way of suitable segmentation algorithms, so that the position and shape of the synovium, cartilage and/or bone are known. Data relating to these anatomical components can now be determined specifically herefrom, for example a thickness of the cartilage, a shape of the synovium, a volume of the bone and the like. A comparison with standard values, for example a standard thickness, can be provided for further analysis. However it is also possible to determine a change, for example to the synovium or cartilage, specifically for the patient currently being treated, by looking at previous image data records, in particular first and second magnetic resonance image data records recorded at an earlier time. It is particularly expedient to have available first and second magnetic resonance image data records recorded before starting the administration of the biological agent, the effectiveness of which is to be assessed for the patient, and to use these as comparison material. It should be noted here that comparisons can also be made from the first and second magnetic resonance image data records 2, 4 themselves, if for example a healthy joint is considered as a comparison.

The result of the analysis in step 5 is at least one first measuring parameter 7. In the present example embodiment Ramris scores are calculated, in respect of synovitis, bone erosion and bone edema.

In the analysis step 6 in the present instance the progression of a contrast agent in the cartilage of the target joint is determined from the magnetic resonance images of the magnetic resonance image data record 4, which were recorded at different times, the time constants of said progression being obtained as second measuring parameters 8, optionally relative to a standard time constant. Such analysis algorithms for perfusion measurements are already known in the prior art and do not have to be examined in detail here.

In a step 9, still as part of the automated measuring protocol covering the entire workflow, the first measuring parameters 7 and the second measuring parameters 8 are combined to determine analysis information 10, in this instance an analysis value, as only the combined consideration of the results of the two imaging methods allows the reliable interpretation in respect of the effectiveness of the biological agent, for example a TNF-α inhibitor. The determination of the analysis information 10 in step 9 therefore supplies an analysis value (score), which by interpreting the analysis value provides a useful aid for a physician when determining the effect of the biological agent, so that it is possible in particular to make a decision concerning the discontinuation of the biological agent.

The analysis information 10, like images of the magnetic resonance image data records 2, 4 and also measuring parameters 7, 8 and other optionally provided analysis results, can be displayed to a user, in particular a physician.

It should also be noted here that it is also possible to use a second magnetic resonance sequence that does not require contrast agent to determine the second magnetic resonance image data record 4, for example by using arterial spin labeling (ASL) methods.

FIG. 2 shows a basic outline of a magnetic resonance facility 11, which is suitable for recording the magnetic resonance image data records 2, 4. Operation of the magnetic resonance facility 11 is controlled by way of a control facility 12, which in the present instance also acts as the inventive analysis device 13. To this end the control facility comprises a computation facility in the form of at least one processor 14, which is connected to at least one storage facility 15. Hardware components can also be provided, which execute parts of the automated analysis in steps 5, 6 or 9. Software components, which are provided for the analysis in steps 5, 6 and 9, are also stored in the storage facility 15, as is a measuring protocol shown as 16, which, as described above, allows the automatic and reproducible performance of embodiments of the inventive method in the control facility 12.

To this end the measuring protocol 16 contains recording parameters of the first and second magnetic resonance sequences as well as corresponding instructions and analysis parameters for the analyses in steps 5, 6 and 9.

Image data, measuring parameters 7, 8 and also the analysis information 10 can be displayed on a display facility 17. An input facility 18 is also provided to receive user inputs, in the case of manual intervention also in respect of embodiments of the inventive method.

As the analysis device 13 the control facility 12 can also have a communication connection 19 to an external system, for example an information system 20, from which data, for example standard values, standard image data records, previous magnetic resonance image data records of the patient can be retrieved but in which analysis information 10 determined can also be stored.

Although the invention has been illustrated and described in detail using the preferred example embodiment, the invention is not limited by the disclosed examples and other variations can be derived therefrom by the person skilled in the art without departing from the scope of protection of the invention.

1. A method for determining analysis information assisting a user assessing the effect of a biological agent for treating a rheumatic disease, after starting administration of the biological agent, the method comprising:
recording a first, morphological magnetic resonance image data record of at least one target joint using a first magnetic resonance sequence;
recording a second magnetic resonance image data record showing the blood flow through the target joint by perfusion imaging using a second magnetic resonance sequence;
determining at least one first measuring parameter relating to the morphology of the joint by analysis of the first magnetic resonance image data record and at least one second measuring parameter describing the blood flow through the target joint by analysis of the second magnetic resonance image data record; and
automatically determining the analysis information from the at least one first and second measuring parameters.

2. The method of claim 1, wherein an analysis value is determined as the analysis information.

3. The method of claim 1, wherein at least one of
the first magnetic resonance sequence used is at least one of a T2-weighted sequence with at least one of fat saturation and a STIR sequence and a gadolinium-enhanced, three-dimensional T1-weighted spin-echo or gradient-echo sequence, and
the second magnetic resonance sequence used is at least one of a sequence that measures the progression of a contrast agent in the cartilage of the target joint and/or a sequence using arterial spin labeling.

4. The method of claim 1, wherein the magnetic resonance sequences are implemented as part of a single measuring protocol.

5. The method of claim 4, wherein at least one of the automatic analysis of the magnetic resonance image data records and the determination of the analysis information also takes place as part of the measuring protocol.

6. The method of claim 1, wherein the first measuring parameter determined is a measuring parameter describing at least one of bone erosion, bone edema, changes to the cartilage and changes to at least one of the synovium and a Ramris score.

7. The method of claim 1, wherein at least one of the first measuring parameter and the second measuring parameter is determined automatically.

8. The method of claim 1, wherein at least one of the following is taken into account for the analysis of the magnetic resonance image data records:
at least one magnetic resonance image data record of the target joint recorded at an earlier time, at least one standard value and a standard image data record.

9. The method of claim 8, wherein a magnetic resonance image data record recorded before starting the administration of the biological agent is used.

10. An analysis device, configured to perform the method of claim 1.

11. The method of claim 2, wherein at least one of
the first magnetic resonance sequence used is at least one of a T2-weighted sequence with at least one of fat saturation and a STIR sequence and a gadolinium-enhanced, three-dimensional T1-weighted spin-echo or gradient-echo sequence, and
the second magnetic resonance sequence used is at least one of a sequence that measures the progression of a contrast agent in the cartilage of the target joint and a sequence using arterial spin labeling.

12. The method of claim 1, wherein at least one of
the first magnetic resonance sequence used is at least one of a T2-weighted sequence with at least one of fat saturation and a STIR sequence for analysis in respect of a measuring parameter describing bone edema, and a gadolinium-enhanced, three-dimensional T1-weighted spin-echo or gradient-echo sequence for analysis in respect of a measuring parameter describing changes to at least one of the synovium and bone erosion, and
the second magnetic resonance sequence used is at least one of a sequence that measures the progression of a contrast agent in the cartilage of the target joint and a sequence using arterial spin labeling.

13. The method of claim 2, wherein at least one of
the first magnetic resonance sequence used is at least one of a T2-weighted sequence with at least one of fat saturation and a STIR sequence for analysis in respect of a measuring parameter describing bone edema, and a gadolinium-enhanced, three-dimensional T1-weighted spin-echo or gradient-echo sequence for analysis in respect of a measuring parameter describing changes to at least one of the synovium and bone erosion, and
the second magnetic resonance sequence used is at least one of a sequence that measures the progression of a contrast agent in the cartilage of the target joint and a sequence using arterial spin labeling.

14. The method of claim 2, wherein the magnetic resonance sequences are implemented as part of a single measuring protocol.

15. The method of claim 14, wherein at least one of the automatic analysis of the magnetic resonance image data records and the determination of the analysis information also takes place as part of the measuring protocol.

16. The method of claim 3, wherein the magnetic resonance sequences are implemented as part of a single measuring protocol.

17. The method of claim 16, wherein at least one of the automatic analysis of the magnetic resonance image data records and the determination of the analysis information also takes place as part of the measuring protocol.

18. The method of claim 7, wherein at least one of the first measuring parameter and the second measuring parameter is determined automatically, based on a segmentation of anatomical structures of the target joint.
